(19) United States
(12) Patent Application Publication
(10) Pub. No.: US 2025/0152790 A1
(43) Pub. Date: May 15, 2025

(54) INDEPENDENTLY-CONTROLLED TWO-SIDED BREAST PUMP

(71) Applicant: Shenzhen TPH Technology Co., Ltd., Shenzhen (CN)

(72) Inventor: Xianzi CHEN, Shenzhen (CN)

(21) Appl. No.: 18/424,412

(22) Filed: Jan. 26, 2024

(30) Foreign Application Priority Data
Nov. 15, 2023 (CN) 2023115222386

(51) Int. Cl. A61M 1/06; A61M 39/22

(52) U.S. Cl. CPC A61M 1/064; A61M 39/223; A61M 2039/226; A61M 2205/3344

(57) ABSTRACT

The present disclosure discloses an independently-controlled two-sided breast pump including a left suction bowl, a right suction bowl and a vacuum pump, wherein the left and right suction bowls communicates with the vacuum pump through left and right three-way valves. The left and right suction bowls communicate with the vacuum pump or with an atmosphere by way of controlling the left and right three-way valves, respectively. The present disclosure achieves independently controlling suction forces on both sides, enhances the stability of the suction force during the pumping process and improves sucked person's comfort.

### CROSS-REFERENCE TO RELATED PATENT APPLICATION

This application claims priority to and the benefit of Chinese Patent Application No. CN202311522238.6 filed in China on Nov. 15, 2023. The disclosure of the above application is incorporated herein in its entirety by reference.

### TECHNICAL FIELD

The present disclosure relates to generally breast pump devices and, more particularly, to an independently-controlled two-sided breast pump.

### BACKGROUND OF THE DISCLOSURE

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Breast-feeding women can use a breast pump in hospitals or at home, of which the function is to pump breast milk out of their breast and lactate with the breast pump to enable relief from congestion. If a mother suffers from mastitis, she can use the breast pump to pump breast milk out of an infected breast, so as to assist herself in a treatment process. The breast pump can relieve pain and rhagade of nipples and expose flat or sunken nipples. In addition, the breast pump allows mothers to provide a complementary form of breastfeeding babies unable to be breast-fed directly because of their inability to suck, cleft lip and palate or premature delivery.

As the traditional two-sided breast pump is put on two-sided breasts, and its master unit cannot separately control a suction force from the cups on the left and right sides, and the suction force is only able to increase or decrease, but unable to be controlled separately on both sides concurrently at the time of adjusting. In addition, when the two-sided breast pump is used for pumping milk on a single-sided breast, it is necessary to plug a unused cup on the other side to avoid air leakage, furthermore, if it is put on the single-sided breast/two-sided breasts in the same setting, a too big suction force will occur at the single-sided breast, so it quite possibly causes damage to the mammary gland, greatly exerting a bad influence on the mother's use experience. The traditional single-sided or two-sided breast pump does not operate under pressure-sensitive control, instead basically depends on a vacuum pump controlling suction time, so as to achieve a certain suction force, but due to the difference in the parameters of the vacuum pump and the time of use, instable suction will occur to the traditional breast pump. The traditional two-sided breast pump can only achieve pumping milk from two-sided breasts in the same pumping mode and the same setting, but cannot get an adjustment according to the volume of milk and the pumping habits of two-sided breasts, greatly affecting the experience of pumping milk and the rate of lactation.

### SUMMARY

In order to solve the problem that the two-sided breast pump cannot independently adjust a single-sided suction force, the present disclosure provides an independently-controlled two-sided breast pump, which communicates with suction bowls on both sides through a vacuum pump and two three-way valves, so as to achieve independently controlling suction forces on both sides.

The technical scheme adopted in the present disclosure is to design an independently-controlled two-sided breast pump comprising a left suction bowl, a right suction bowl and a vacuum pump, wherein the left suction bowl communicates with the vacuum pump through a connecting tube and a left three-way valve, so as to enable the left suction bowl to communicate with the vacuum pump or with an atmosphere by way of controlling the left three-way valve; the right suction bowl communicates with the vacuum pump through a connecting tube and a right three-way valve, so as to enable the right suction bowl to communicate with the vacuum pump or with an atmosphere by way of controlling the right three-way valve.

Preferably, both the left three-way valve and the right three-way valve are an electric control valve, and the left three-way valve and the right three-way valve are independently controlled by means of an electronic controller of the master unit, respectively.

Preferably, the present disclosure further includes a left pressure sensor used to detect air pressure in the left suction bowl, and a right pressure sensor used to detect air pressure in the right suction bowl, the electronic controller controls the operation of the left three-way valve according to the information of the left pressure sensor, and the electronic controller controls the operation of the right three-way valve according to the information of the right pressure sensor.

Preferably, the electronic controller controls the operation of the left three-way valve and the right three-way valve by means of impulse voltage.

Preferably, the pulse voltage is PWM (pulse width modulation) voltage or PFM (pulse frequency modulation) voltage.

In an embodiment, the two-sided breast pump further includes a left milk-collecting cup and a right milk-collecting cup, the left suction bowl and the right suction bowl are set inside the left milk-collecting cup and the right milk-collecting cup, respectively.

In an embodiment, the electronic controller controls the switching frequency of the left three-way valve by means of a first impulse voltage signal, so as to control pressure in the left suction bowl, and the electronic controller controls the switching frequency of the right three-way valve by means of a second impulse voltage signal, so as to control pressure in the right suction bowl, the first impulse voltage signal and the second impulse voltage signal independently control the left three-way valve and the right three-way valve, respectively.

In an embodiment, the vacuum pump has a constant voltage for control.

In an embodiment, the amplitude of the pulse voltage of the first pulse voltage signal is the same as that of the second pulse voltage signal.

In an embodiment, the pulse width and/or pulse frequency of the first impulse voltage signal is different from that of the second impulse voltage signal.

Compared with the prior art, the present disclosure has the following beneficial effects:

In the present disclosure, the vacuum pump communicates with the suction bowl on both sides through the three-way valves, respectively, so as to achieve independently controlling suction forces on both sides, and independently controlling a suction force on each side during using the two-sided breast pump, furthermore efficiently monitoring suction changes in real-time during the pumping process. Whether it is used in a single-sided or two-sided mode, it can be controlled separately and capable of accurately controlling size of a suction force, enhancing the stability of the suction force during the pumping process and improving sucked person's comfort and body feeling during use. Only one vacuum pump is used with two independent three-way valves to achieve two-sided independent control, instead of two vacuum pumps used to achieve independent control on both sides, so it saves the cost of vacuum pumps.

### BRIEF DESCRIPTION OF THE DRAWINGS

We shall describe the present disclosure in detail in combination with specific examples and drawings, as follows.

FIG. 1 is a schematic diagram showing one example of the present disclosure, wherein the left and right three-way valves are in an open state.

FIG. 2 is a schematic diagram of the example shown in FIG. 1, wherein the left and right three-way valves in a closed state.

FIG. 3 shows a comparison of the impulse voltages in a mode of synchronously pumping milk on two-sided breasts.

FIG. 4 shows a comparison of the impulse voltages in a mode of asynchronously pumping milk on two-sided breasts.

FIG. 5 shows a comparison of the impulse voltages in a mode of pumping milk independently from respective two-sided breasts at different frequencies and pressures.

FIG. 6 is a physical picture of the two-sided breast pump.

FIG. 7 is a schematic diagram of only pumping milk from a single-sided right breast.

Where, 1—left suction bowl; 2—right suction bowl; 3—vacuum pump; 4—left three-way valve; 5—right three-way valve; 6—left pressure sensor; 7—right pressure sensor; 8—left milk-collecting cup; 9—right milk-collecting cup.

### DETAILED DESCRIPTION OF SOME EMBODIMENTS

We shall explain the principle and structure of the present disclosure in detail in combination with the drawings and specific examples as follows, but the technical solution provided by the present disclosure is not limited to these examples.

### Example 1

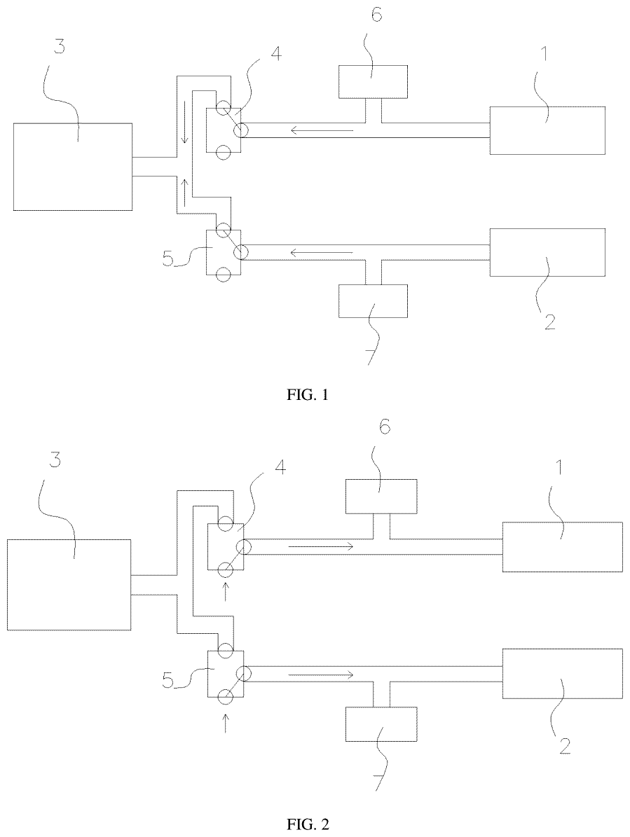

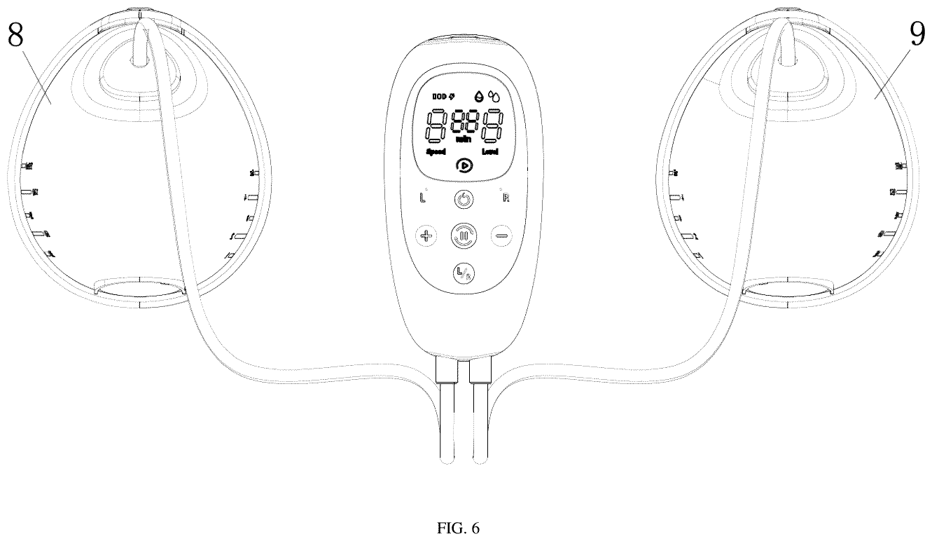

As shown in FIG. 1 and 6, the two-sided breast pump provided by the present disclosure includes the left milk-collecting cup 8, the right milk-collecting cup 9 and the vacuum pump 3. The left suction bowl 1 and right suction bowl 2 are set inside the left milk-collecting cup and the right milk-collecting cup, respectively, the left suction bowl and the right suction bowl communicates with left and right three-way valves through a sealing plug-in port and a connecting tube arranged on the left and right milk-collecting cups, and the left and right three-way valves communicates with the vacuum pump 3 through the connecting tube, which may be made of silicone.

The milk-collecting cup includes a silicone bell mouth, a suction bowl, a milk collection chamber and a sealing plug-in port. A unidirectional valve and a draining port used to drain the suctioned milk are arranged on the bottom of the milk-collecting cup. The suction bowl, also called a sucker, is part of the breast pump that is used to cover the breast for pumping milk. The suction bowl is usually made of a soft silicone or rubber material, ensuring a comfortable fit on mother's breasts while providing a suction force.

The two-sided breast pump also includes a master unit on which two separate plug-in ports and displayers are arranged, as well as several buttons used to switch between modes of pumping milk from left and right sides and control them. The silicone connecting tube connects the left and right milk-collecting cups with the master unit by way of plugging. The vacuum pump, the left and right three-way valves, a pressure sensing module, a micro-control unit and a battery are arranged inside the main unit.

The left suction bowl 1 communicates with the left three-way valve 4 through a connecting tube, the left three-way valve communicates with the vacuum pump 3 through a connecting tube, thus the left three-way valve 4 is operated to enable the left suction bowl 1 to communicate with vacuum pump 3 or with an atmosphere. In the same way, the right suction bowl 2 communicates with the right three-way valve 5 through a connecting tube, the right three-way valve communicates with the vacuum pump 3 through a connecting tube, thus the right three-way valve 5 is operated to enable the right suction bowl 2 to communicate with the vacuum pump 3 or with an atmosphere. The left three-way valve 4 and the right three-way valve 5 are the same in structure, and each has three ports, of which two ports communicate with the suction bowl and the vacuum pump 3, respectively, and the other port communicates with an atmosphere, so as to switch the communication between the suction bowl 3 and the vacuum pump 3 or an atmosphere by way of controlling the movement of an inside valve core. When the valve core opens, the suction bowl communicates with the vacuum pump 3 and disconnects from the atmosphere; when the valve core closes, the suction bowl communicates with the atmosphere, and the port that communicates with the vacuum pump 3 closes. By way of controlling the left three-way valve and the right three-way valve, respectively, the left and right suction bowls can independently get control of sucking and size of a suction force, and they do not influence each other. The present disclosure adopts the mode of a single vacuum pump plus two three-way valves that can effectively reduce costs, improve battery life and concurrently have functional diversity, applicability and convenience. In the case of only pumping milk from a single-sided breast, it is achievable just to directly insert the connecting tube into the connecting port on this side, and there is no need to block the connecting port on the other side, greatly improving the convenience of use. In this way, only one vacuum pump 3 is used with two independent three-way valves to realize independent control on both sides, instead of two vacuum pumps used to achieve independent control on both sides, so it saves the cost of vacuum pumps.

The left three-way valve 4 and the right three-way valve 5 are all electric control valves, such as electromagnetic three-way valves, which are independently controlled by the electronic controller on the master unit, respectively, for example, they may be controlled by a MCU control module. For the electromagnetic three-way valves, on being electrified they open, on being dis-electrified they close.

Pressure sensors are respectively arranged on the connecting tubes between the left and right suction bowls and the left and right three-way valves; thus, they are used to detect the pressure in the suction bowls. The left pressure sensor 6 is used for detecting the air pressure in the left suction bowl 1, and the right pressure sensor 7 is used for detecting the air pressure in the right suction bowl 2. The electronic controller of the master unit controls the operation state of the left three-way valve 4 according to the information of the left pressure sensor 6, and controls the operation state of the right three-way valve 5 according to the information of the right pressure sensor 7, so that the suction pressure of the suction bowl is in a set pressure. When the pressure of one side of the suction bowl is lower than the set pressure, the electronic controller of the master unit can control the three-way valve of this side to open, so as to enable the suction bowl to communicate with the vacuum pump 3, and when the pressure of one side of the suction bowl is greater than the set pressure, the three-way valve of this side can be controlled to close, so that the suction bowl of this side communicates with the atmosphere. The pressure-sensitive module ensures that the suction force is basically constant every time, improving the comfort of use and avoiding an excessive suction force from injuring the breast. The master unit monitors the suction value of the two-sided breast pump in real time, and can keep a basically constant suction value regardless of single-sided or two-sided use, improving the control accuracy of the strength for pumping milk.

The electronic controller controls the operation of the left three-way valve 4 and the right three-way valve 5 by means of impulse voltage, so as to quickly switch the communication state. The pulse voltage may be PWM (pulse width modulation) voltage or PFM (pulse frequency modulation) voltage, so as to achieve stably and flexibly adjusting pressure.

The electromagnetic three-way valve can be electrified or dis-electrified under the control of PWM voltage, so as to control the electromagnetic three-way valve to open and close, on opening the vacuum pump 3 will communicate with the suction bowl and perform suction to form a negative pressure, so as to pump milk from the breast; on closing the vacuum pump 3 disconnects from the communication, thus the suction bowl will communicate with the atmosphere, so it will not produce a negative pressure and stops pumping milk at this moment.

Milk-collecting cups are arranged on both the left suction bowl 1 and the right suction bowl 2, so that the suctioned milk enters the respective milk-collecting cups for storage.

During operation, the electronic controller of the master unit controls the switching frequency of the left three-way valve 4 by means of a first impulse voltage signal, so as to control the pressure change in the left suction bowl 1, and the electronic controller of the master unit controls the switching frequency of the right three-way valve 5 by means of a second impulse voltage signal, so as to control the pressure change in the right suction bowl 2. The first impulse voltage signal and the second impulse voltage signal independently control the left three-way valve 4 and the right three-way valve 5, respectively. The first impulse voltage signal and the second impulse voltage signal may be identical with or different from each other, and they can be controlled independently, but do not influence each other.

The control voltage of the vacuum pump 3 is constant, and the pressure control of the suction bowl on this side gets controlled only through the three-way valve on each side.

The amplitude of the pulse voltage of the first pulse voltage signal is the same as that of the second pulse voltage signal, and the three-way valve connects and disconnect only under the control of the pulse width or frequency, so as to control the change of suction. The pulse width and/or pulse frequency of the first impulse voltage signal is different from that of the second impulse voltage signal, and both those can be controlled independently.

During pumping milk form two-sided breasts, the left three-way valve 4 controls pumping milk on the left side, but it will not be influenced by the operation state of the right three-way valve, that is, no matter whether the right side is in a state of vacuuming for pumping milk, or a state of deflating for stopping pumping milk, it will not influence the operation of pumping milk on the left side. In the same way, pumping milk on the left side will not influence the operation of pumping milk on the right side. The operations of pumping milk on the left and right sides are completely independent and do not influence each other.

When mothers use the breast pump provide by the present disclosure, they can independently adjust the mode and strength of pumping milk completely according to the difference of their own body constitutions and the difference in the milk volume of their left and right breasts, so that the left and right breasts can be adjusted as a mode of concurrently pumping milk, alternately pumping milk, or pumping milk from a single-sided breast and other modes, without being limited to a single mode of pumping milk.

As shown in FIG. 6, there are multiple buttons on the master unit for selecting and controlling kinds of modes for pumping milk on the left and right sides, and the displayer on the master unit can respectively show the states of pumping milk on the left and right sides. When the button switches to the state of pumping milk on the left side and display it, the electronic controller controls the left three-way valve to operate, and when the button switches to the state of pumping milk on the right side and display it, the electronic controller controls the right three-way valve to operate, thus the two control modes are independent of each other and do not interfere with each other.

The above-mentioned independent control means that, regardless of on which side the suction bowl is in the state of pumping milk, or is in the state of not pumping milk, it will not influence the operation state of the suction bowl on the other side, and on the other side pumping milk or stopping pumping still can go as usual.

The displayer can switch the operation state of pumping milk on the left or right side by way of pressing the L/R button.

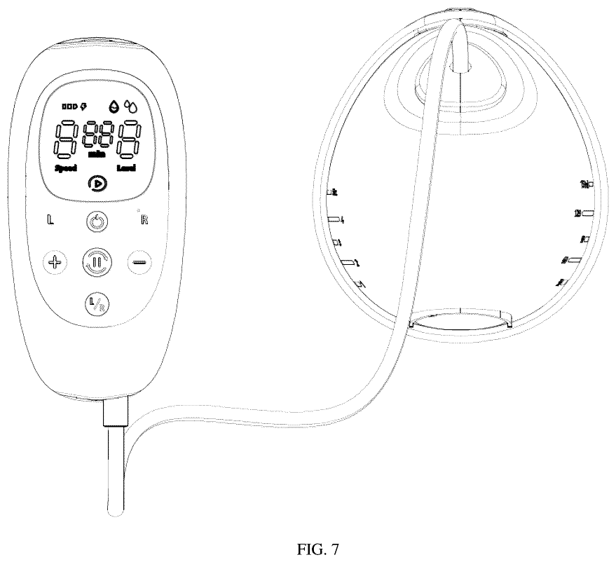

FIG. 7 is a schematic diagram of only pumping milk from a single-sided right breast. On using it the R indicator lights up and the L indicator goes out, and there is no need to block the plug-in port of the silicone tube.

FIG. 1 shows that the left and right three-way valves are in an open state, at this time, the left and right suction bowls are connected to the vacuum pump for the mode of pumping milk.

FIG. 2 shows that the left and right three-way valves in a closed state, at this time, the left and right suction bowls are not connected to the vacuum pump for the mode of stopping pumping milk.

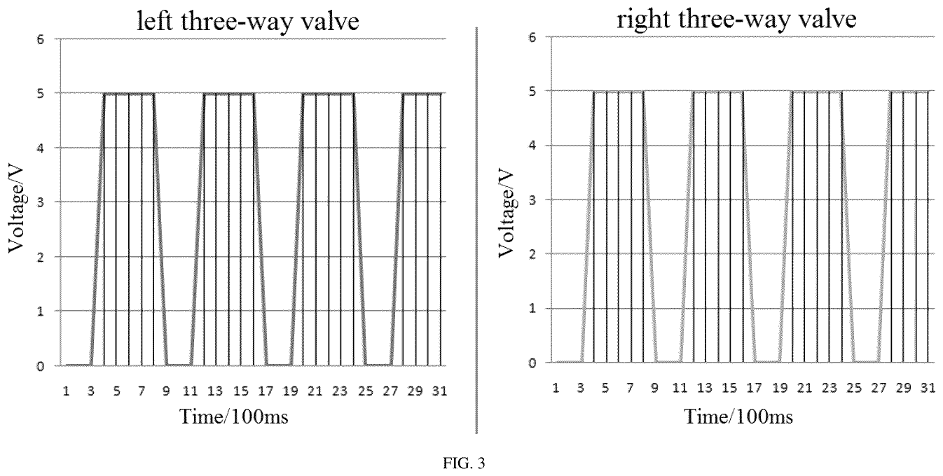

FIG. 3 shows a comparison of the impulse voltages acting on the left and right three-way valves in a mode of synchronously pumping milk and adopting the same frequency and suction force on two-sided breasts.

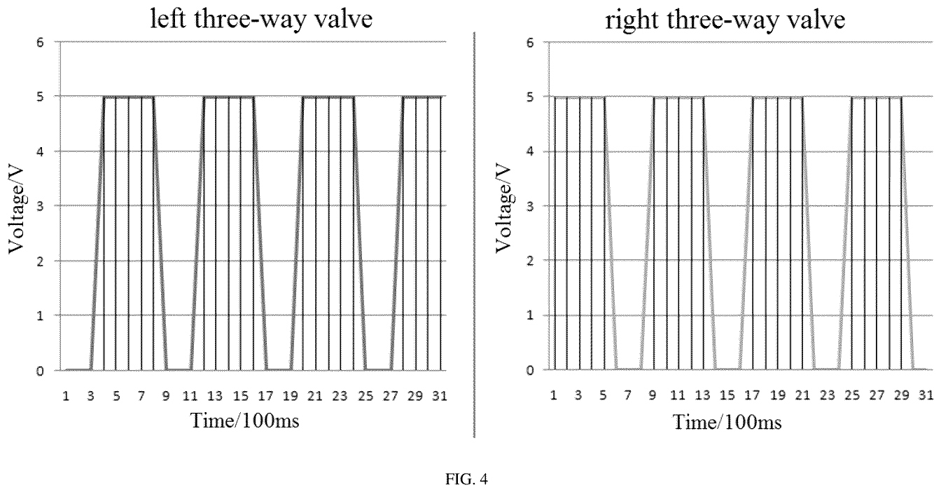

FIG. 4 shows a comparison of the impulse voltages acting on the left and right three-way valves in a mode of asynchronously pumping milk and adopting the same frequency and suction force on two-sided breasts.

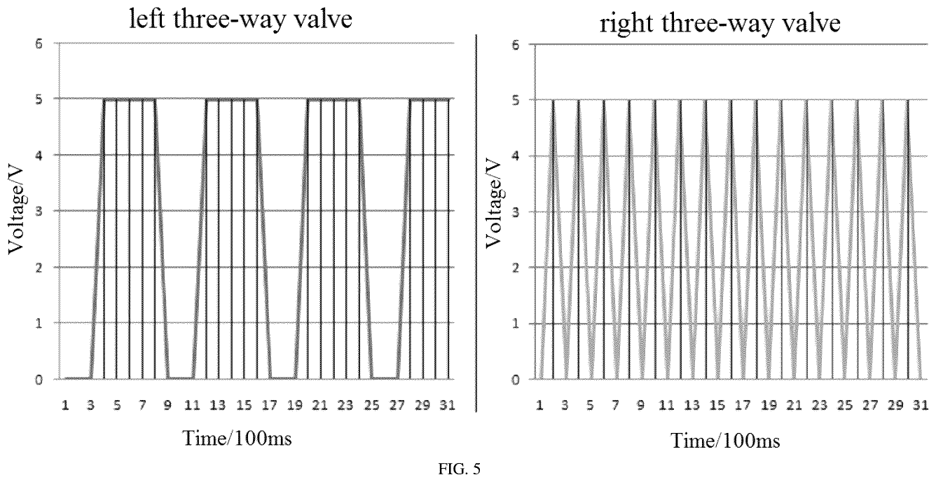

FIG. 5 shows a comparison of the impulse voltages acting on the left and right three-way valves in a mode of pumping milk independently from respective two-sided breasts at different frequencies and pressures.

The specific embodiments described herein only give some examples for the conception of the present disclosure and do not impose a limitation on the present disclosure. All modifications, additions or substitutions made within the present disclosure shall fall within in the protection scope defined in the claims of the present disclosure.

What is claimed is:

1. An independently-controlled two-sided breast pump comprising a left suction bowl, a right suction bowl and a vacuum pump, wherein said left suction bowl communicates with said vacuum pump through a connecting tube and a left three-way valve, so as to enable said left suction bowl to communicate with said vacuum pump or with an atmosphere by way of controlling said left three-way valve; said right suction bowl communicates with said vacuum pump through a connecting tube and a right three-way valve, so as to enable said right suction bowl to communicate with said vacuum pump or with an atmosphere by way of controlling said right three-way valve.

2. The independently-controlled two-sided breast pump according to claim 1, wherein it further comprises a master unit, both said left three-way valve and said right three-way valve are an electric control valve, and said left three-way valve and said right three-way valve are independently controlled by means of an electronic controller of said master unit, respectively.

3. The independently-controlled two-sided breast pump according to claim 2, wherein it further comprises a left pressure sensor used to detect air pressure in said left suction bowl, and a right pressure sensor used to detect air pressure in said right suction bowl, said electronic controller controls the operation of said left three-way valve according to the information of said left pressure sensor, and said electronic controller controls the operation of said right three-way valve according to the information of said right pressure sensor.

4. The independently-controlled two-sided breast pump according to claim 2, wherein said electronic controller controls the operation of said left three-way valve and said right three-way valve by means of impulse voltage.

5. The independently-controlled two-sided breast pump according to claim 4, wherein said pulse voltage is PWM (pulse width modulation) voltage or PFM (pulse frequency modulation) voltage.

6. The independently-controlled two-sided breast pump according to claim 1, wherein it further comprises a left milk-collecting cup and a right milk-collecting cup, said left suction bowl and said right suction bowl are set inside said left milk-collecting cup and said right milk-collecting cup, respectively.

7. The independently-controlled two-sided breast pump according to claim 2, wherein said electronic controller controls the switching frequency of said left three-way valve by means of a first impulse voltage signal, so as to control pressure in said left suction bowl, and said electronic controller controls the switching frequency of said right three-way valve by means of a second impulse voltage signal, so as to control pressure in said right suction bowl, said first impulse voltage signal and said second impulse voltage signal independently control said left three-way valve and said right three-way valve, respectively.

8. The independently-controlled two-sided breast pump according to claim 7, wherein said vacuum pump has a constant voltage for control.

9. The independently-controlled two-sided breast pump according to claim 7, wherein the amplitude of the pulse voltage of said first pulse voltage signal is the same as that of said second pulse voltage signal.

10. The independently-controlled two-sided breast pump according to claim 7, wherein the pulse width and/or pulse frequency of said first impulse voltage signal is different from that of said second impulse voltage signal.
